(19) United States
(12) Patent Application Publication
(10) Pub. No.: US 2011/0245925 A1
(43) Pub. Date: Oct. 6, 2011

(54) ALLOGRAFT SPACER FILLERS

(76) Inventors: Gina Lee, Edison, NJ (US); John Halkiadakis, Bridgewater, NJ (US)

(21) Appl. No.: 13/049,542

(22) Filed: Mar. 16, 2011

Related U.S. Application Data
(60) Provisional application No. 61/320,816, filed on Apr. 5, 2010.

(51) Int. Cl. A61F 2/44

(52) U.S. Cl. 623/17.16

(57) ABSTRACT

An allograft spacer filler for filling a medullary canal of a spinal spacer. The spacer filler is adapted to be hydrated with bone marrow aspirate (BMA). Allograft spacer filler constructs are created out of fully demineralized cancellous tissue. The spacer fillers are used in bone grafting procedures in combination with BMA. In other words, the spacer fillers provide an environment in which cells can be added (i.e. autologous or allogenic cells). In addition, the spacer fillers expand upon rehydration and provide a maximum surface area for graft to endplate contact in the spine. The spacer fillers provide a matrix for incorporation of new bone, have shape memory, and are sized and shaped such that they fit within a lumbar spacer, other allograft spinal implants, or can be used in other applications where an allograft is used.

### CROSS-REFERENCE TO RELATED APPLICATIONS

This application claims the benefit of U.S. Provisional Patent Application Ser. No. 61/320,816, filed Apr. 5, 2010, the disclosure of which is incorporated by reference herein in its entirety.

### FIELD OF THE INVENTION

The present invention relates to surgical implants and, more particularly, to allograft fillers for spinal spacers.

### BACKGROUND OF THE INVENTION

There is a need for an allograft spacer filler for filling a medullary canal of a spinal spacer, and which is capable of being hydrated with hydrating materials, such as bone marrow aspirate (BMA). Current technology provides bony structures without cells. There are no commercially available matrices that have distinct shapes with the capability to absorb BMA. Empirical evidence has shown that adult stem cells found in BMA can potentially accelerate bone healing.

### SUMMARY OF THE INVENTION

An allograft spacer filler for filling a medullary canal of a spinal spacer. The spacer filler is adapted to be hydrated with bone marrow aspirate (BMA). Allograft spacer filler constructs are created out of fully demineralized cancellous tissue. The spacer fillers are used in bone grafting procedures in combination with BMA. In other words, the spacer fillers provide an environment in which cells can be added (i.e. autologous or allogenic cells). In addition, the spacer fillers expand upon rehydration and provide a maximum surface area for graft to endplate contact in the spine. The spacer fillers provide a matrix for incorporation of new bone, have shape memory, and are sized and shaped such that they fit within a lumbar spacer, other allograft spinal implants, or can be used in other applications where an allograft is used. Combining BMA with an allograft spacer filler material provides an enhancement of bioactivity for vascular growth and new bone formation to the allograft matrix.

In an embodiment, the allograft spacer fillers are used for bony voids or defects that are not intrinsic to the stability of the bony structure in the extremities, spine and pelvis. The allograft spacer fillers are packed or placed into the medullary cavity of an allograft spine spacer and may be combined with autogenous blood, bone marrow, saline, and/or autologous or allogenic cells.

### BRIEF DESCRIPTION OF THE DRAWINGS

Reference is made to the following detailed description of the exemplary embodiments considered in conjunction with the accompanying drawings, in which:

FIGS. 1A through 1C show views of an embodiment of an allograft spacer filler;

FIGS. 2A through 2B show views of another embodiment of an allograft spacer filler;

FIGS. 3A through 3C show views of another embodiment of an allograft spacer filler;

FIGS. 4A through 4B show views of another embodiment of an allograft spacer filler;

FIGS. 5A through 5C show views of another embodiment of an allograft spacer filler;

FIGS. 6A through 6I show views of another embodiment of an allograft spacer filler;

FIGS. 7A through 7C show views of another embodiment of an allograft spacer filler;

FIGS. 8A through 8C show views of another embodiment of an allograft spacer filler; and

FIGS. 9A through 9C show views of another embodiment of an allograft spacer filler.

### DETAILED DESCRIPTION OF THE DRAWINGS

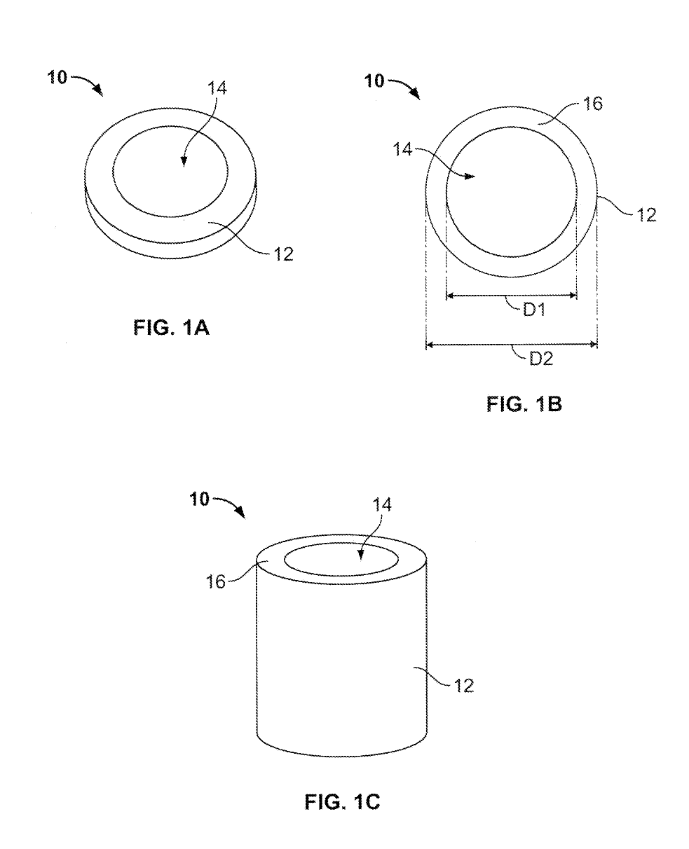

Referring to FIGS. 1A through 1C, an allograft spacer filler 10 includes an elongated, cylindrical-shaped body 12 and a circular-shaped recess 14 formed within one end 16 of the body 12. The spacer filler 10 is made from fully demineralized cancellous tissue. The recess 14 is adapted to be filled with demineralized bone matrix (DBM), bone marrow aspirate (BMA), or any other type of hydrating agent. Once filled, the spacer filler 10 is placed inside a medullary canal of an allograft spine spacer (not shown in the Figures). In an embodiment, the spacer filler 10 has an inside diameter D1 within a range of approximately 6 mm to approximately 13 mm, an outside diameter D2 in a range of approximately 10 mm to approximately 17 mm, a height in a range of 13 mm to 17 mm, and a depth of the recess 14 in a range of approximately 11 mm to approximately 15 mm. In other embodiments, the spacer filler 10 consists of other shapes and sizes.

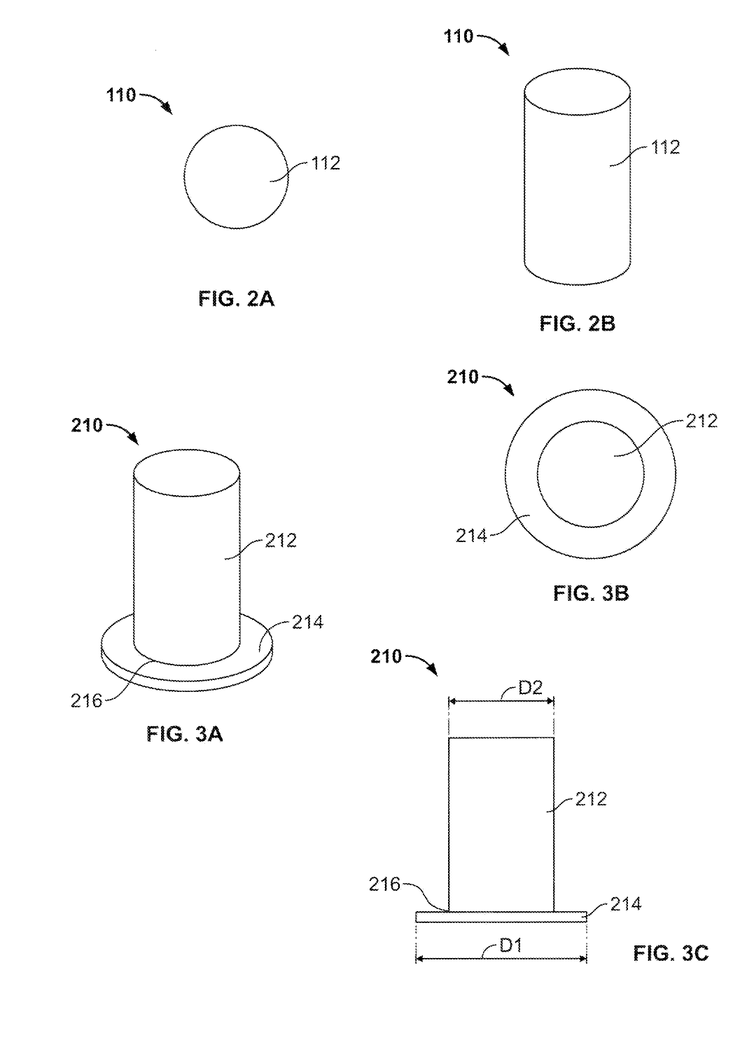

Referring to FIGS. 2A and 2B, an allograft spacer filler 110 includes an elongated, cylindrical-shaped body 112. The spacer filler 110 is made from fully demineralized cancellous tissue, and is adapted to be hydrated with demineralized bone matrix (DBM), bone marrow aspirate (BMA), or any other type of hydrating agent. In an embodiment, the spacer filler 110 fits snugly inside a medullary canal of an allograft spine spacer (not shown in the Figures). In another embodiment, the spacer filler 110 is sized and shaped for insertion within the recess 14 of the spacer filler 10 described above, and functions as a “cap” (not shown in the Figures). In an embodiment, the spacer filler 110 includes a diameter within a range of approximately 10 mm to approximately 17 mm, and a height within a range of approximately 13 mm to approximately 17 mm. In other embodiments, the spacer filler 110 consists of other shapes and sizes.

Referring to FIGS. 3A through 3C, an allograft spacer filler 210 includes an elongated, cylindrical-shaped body 212 and a head 214 formed at one end 216 of the body 212. The spacer filler 210 is made from fully demineralized cancellous tissue, and is adapted to be hydrated with demineralized bone matrix (DBM), bone marrow aspirate (BMA), or any other type of hydrating agent. The spacer filler 210 fits inside a medullary canal of an allograft spine spacer (not shown in the Figures). In an embodiment, the diameter D1 of the head 214 is larger than a diameter of the medullary canal of the spine spacer. In this case, the body 212 fills the medullary canal, while the head 214 rests on one side (top or bottom) of the spacer and stops the spacer filler 210 from falling through the spacer, even if a diameter D2 of the body 212 is smaller than the diameter of the medullary canal. In another embodiment, the spacer filler 210 is sized and shaped for insertion within the recess 14 of the spacer filler 10 described above, and acts as a “cap” (not shown in the Figures). That is, the body 212 of the spacer filler 210 is inserted into the recess 14 of the spacer filler 10, and the head 214 of the spacer filler 210 “caps” the spacer filler 10. In an embodiment, the head 214 includes the diameter D1 within a range of approximately 10 mm to approximately 17 mm and a height of approximately 1.5 mm±0.5 mm, while the body 212 includes the diameter D2 within a range of approximately 8 mm to approximately 15 mm, and a height within a range of approximately 13 mm to approximately 17 mm. In other embodiments, the spacer filler 210 can consist of different shapes and sizes.

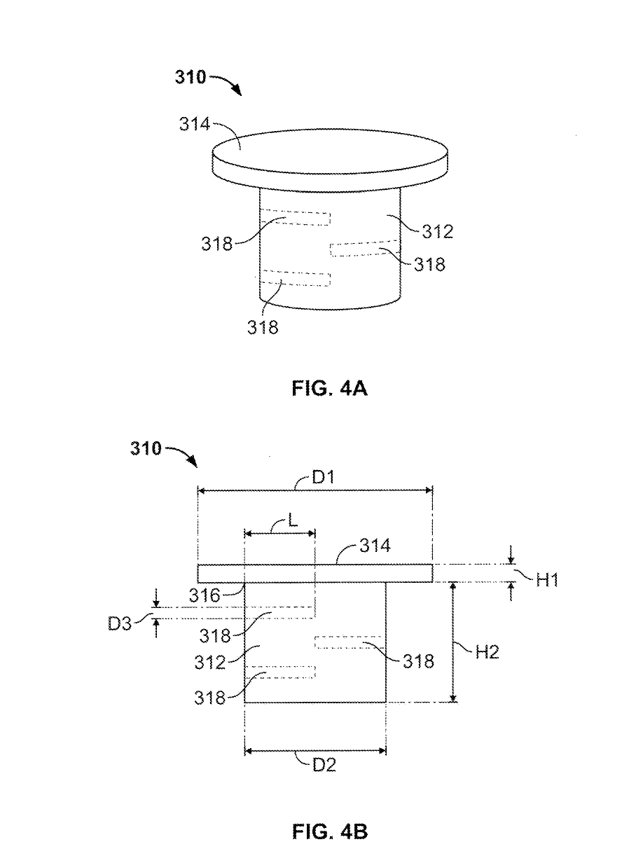

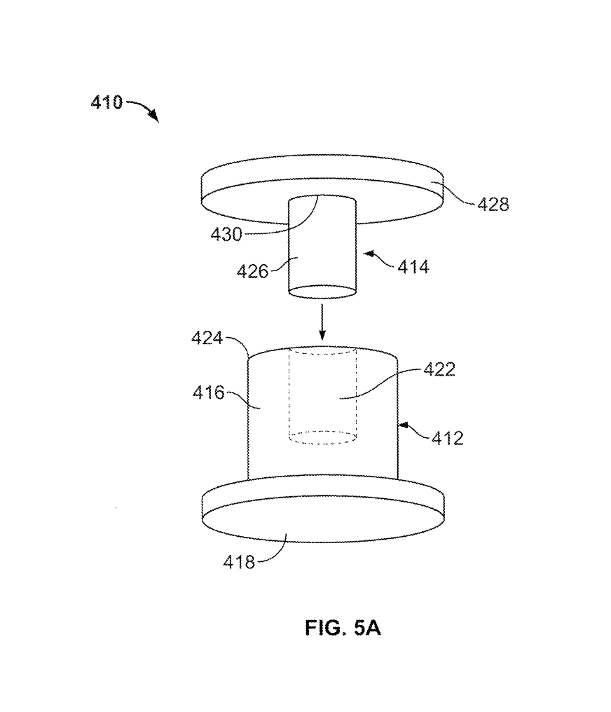

Referring to FIGS. 4A and 4B, an allograft spacer filler 310 includes an elongated, cylindrical-shaped body 312 and a head 314 formed at one end 316 of the body 312. A plurality of holes 318 are formed (e.g., drilled, bored, etc.) within the side of the body 312. The holes 318 may be positioned in any desired pattern within the side of the body 312 (e.g., staggered around the body 312, aligned horizontally or vertically along the length of the body 312, etc.). In an embodiment, the spacer filler 310 includes three of the holes 318, but it may include more or less than three of the holes 318. The spacer filler 310 is made from fully demineralized cancellous tissue, and is adapted to be hydrated with demineralized bone matrix (DBM), bone marrow aspirate (BMA), or any other type of hydrating agent. The holes 318 provide for greater porosity of and faster demineralization time for the spacer filler 310. The spacer filler 310 fits inside a medullary canal of an allograft spine spacer (not shown in the Figures). In an embodiment, the diameter D1 of the head 314 is larger than a diameter of the medullary canal of the spine spacer. In this case, the body 312 fills the medullary canal, while the head 314 rests on one side (top or bottom) of the spacer and stops the spacer filler 310 from falling through the spacer, even if a diameter D2 of the body 312 is smaller than the diameter of the medullary canal. In another embodiment, the spacer filler 310 is sized and shaped for insertion within the recess 14 of the spacer filler 10 described above, and act as a “cap” (not shown in the Figures). That is, the body 312 of the spacer filler 310 is inserted into the recess 14 of the spacer filler 10, and the head 314 of the spacer filler 310 “caps” the spacer filler 10. In an embodiment, the head 314 includes the diameter D1 within a range of approximately 10 mm to approximately 17 mm and a height H1 of approximately 1.5 mm±0.5 mm, while the body 312 includes the diameter D2 within a range of approximately 8 mm to approximately 15 mm, and a height H2 within a range of approximately 13 mm to approximately 17 mm. In other embodiments, the spacer filler 310 can consist of different shapes and sizes. In an embodiment, each of the holes 318 includes a diameter D3 of approximately 1.5 mm±0.5 mm and a depth (length) L within a range of approximately 4 mm to approximately 7 mm. In other embodiments, the holes 318 can consist of different shapes and sizes.

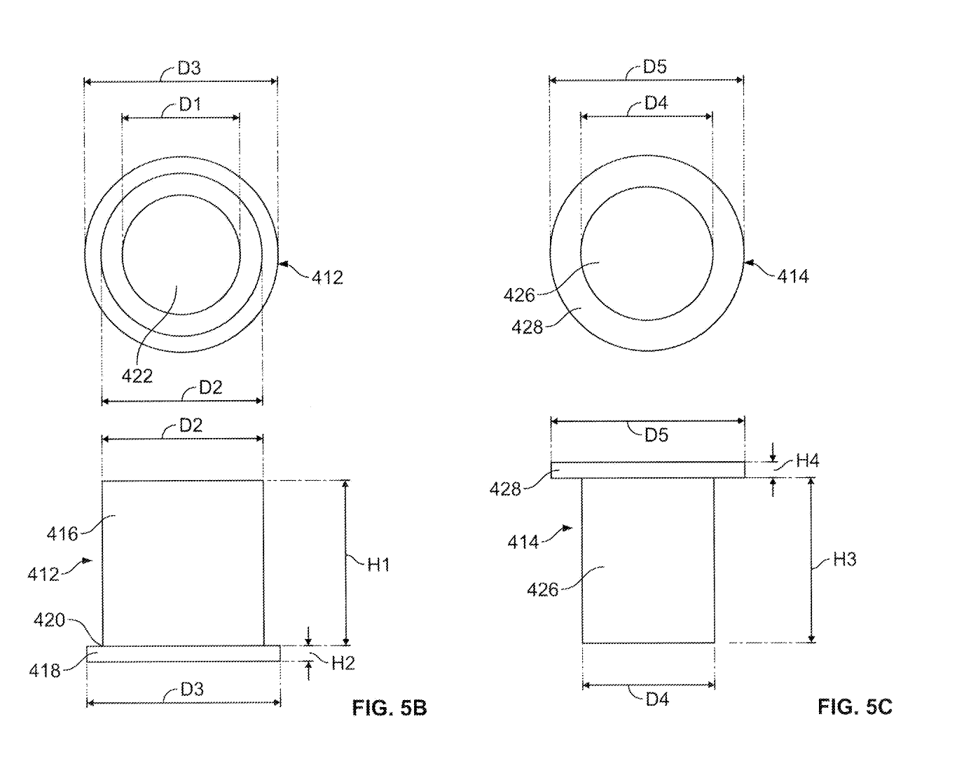

Referring to FIGS. 5A through 5C, an allograft spacer filler 410 includes a cup 412 and a cap 414 that mates with the cup 412. The cup 412 includes an elongated, cylindrical-shaped body 416, a head 418 formed at one end 420 of the body 416, and a circular-shaped recess 422 formed within an opposite end 424 of the body 416. The cap 414 includes an elongated, cylindrical-shaped body 426 and a head 428 formed at one end 430 of the body 426. The spacer filler 410 is made from fully demineralized cancellous tissue. The recess 422 of the cup 412 is adapted to be filled with demineralized bone matrix (DBM), bone marrow aspirate (BMA), or any other type of hydrating agent. Once the cup 412 is filled, it is capped by the cap 414, i.e., the body 426 of the cap 414 is inserted into the recess 422 of the cup 422. The spacer filler 410 is placed inside a medullary canal of an allograft spine spacer (not shown in the Figures). In an embodiment, both the diameter D5 of the head 428 of the cap 414 and the diameter D3 of the head 418 of the cup 412 are larger than the diameter of the medullary canal of the spine spacer. In this case, the body 416 of the cup 412 and the body 426 of the cap 414 fill the medullary canal, while the head 428 of the cap 414 rests on one side of the spacer (top or bottom) and the head 418 of the cup 412 rests on an opposite side of the spacer, so that even if the diameter D2 of the body 416 of the cup 412 and the diameter D4 of the body 426 of the cap 414 are each smaller than the diameter of the medullary canal, the spacer filler 410 will not fall out. In an embodiment, the body 416 of the cup 412 includes an inner diameter D1 within a range of approximately 6 mm to approximately 13 mm, an outer diameter D2 within a range of approximately 10 mm to approximately 17 mm, and a height H1 in a range of approximately 13 mm to approximately 17 mm, while the recess 422 of the cup 412 has a depth in a range of approximately 11 mm to approximately 15 mm. The head 418 of the cup 412 includes a diameter D3 in a range of approximately 12 mm to approximately 19 mm and a height H2 of approximately 1.5 mm±0.5 mm. In an embodiment, the body 426 of the cap 414 has a diameter D4 in a range of approximately 6 mm to approximately 13 mm, and a height H3 in a range of approximately 11 mm to approximately 15 mm, while the head 428 of the cap 414 has a diameter D5 in a range of approximately 12 mm to approximately 19 mm, and a height H4 of approximately 1.5±0.5 mm. In other embodiments, the cup 412 and the cap 414 can consist of different shapes and sizes.

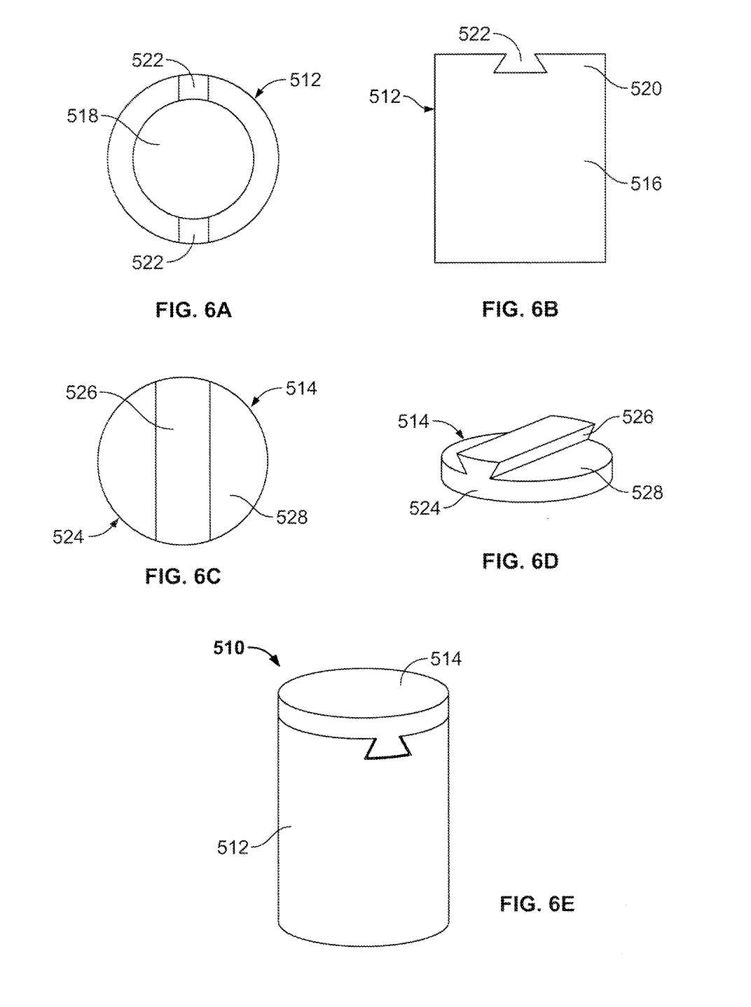

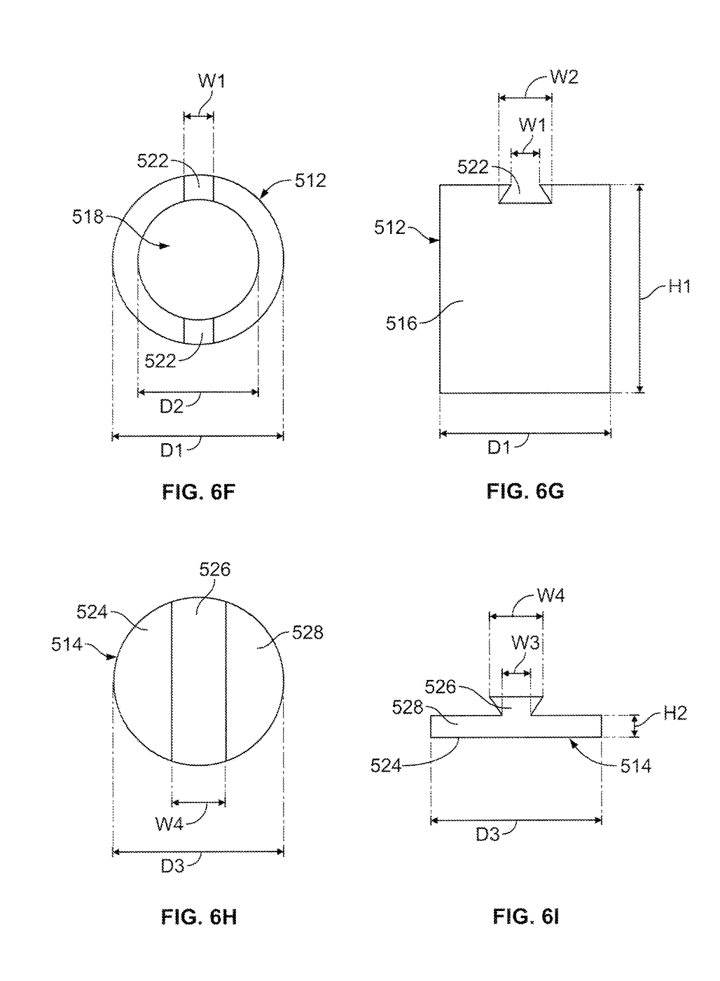

Referring to FIGS. 6A through 6I, an allograft spacer filler 510 includes a cup 512 and a cap 514 that mates with the cup 512. The cup 512 includes an elongated, cylindrical-shaped body 516, a circular-shaped recess 518 formed within one end 520 of the body 516, and a pair of diametrically opposed, trapezoidal-shaped slots 522 formed within the end 520 of the body 516. The cap 514 includes a circular-shaped base 524 and a trapezoidal-shaped tab 526 extending from a surface 528 of the base 524. The tab 526 of the cap 514 is sized and shaped to engage and interlock with the slots 522 of the cup 512. While the slots 522 and the tab 526 are each trapezoidal in shape, they can consist of other shapes and sizes. The spacer filler 510 is made from fully demineralized cancellous tissue. The recess 518 of the cup 512 is adapted to be filled with demineralized bone matrix (DBM), bone marrow aspirate (BMA), or any other type of hydrating agent. Once the cup 512 is filled, it is capped by the cap 514, such that the tab 526 of the cap 514 engages and interlocks with the slots 522 of the cup 512, thereby securing the cap 514 to the cup 512 and keeping the contents within the cup 512 secure. The spacer filler 510 is placed inside a medullary canal of an allograft spine spacer (not shown in the Figures). In an embodiment, the cup 512 includes an outer diameter D1 in a range of approximately 10 mm to approximately 17 mm, an inner diameter D2 in a range of approximately 6 mm to approximately 13 mm, and a height H1 in a range of approximately 11 mm to approximately 15 mm, while the recess 518 of the cup 512 has a depth in a range of approximately 9 mm to approximately 13 mm. In an embodiment, each of the slots 522 has a top width W1 of approximately 2 mm±0.5 mm and a base width W2 of approximately 5 mm±0.5 mm. In an embodiment, the cap 514 includes a diameter D3 in a range of approximately 10 mm to approximately 17 mm and a height H2 in a range of approximately 1 mm to approximately 2 mm, while the tab 526 has a top width W3 of approximately 2 mm±0.5 mm and a base width W4 of approximately 5 mm±0.5 mm. In other embodiments, the cup 512 and the cap 514 can each consist of different shapes and sizes.

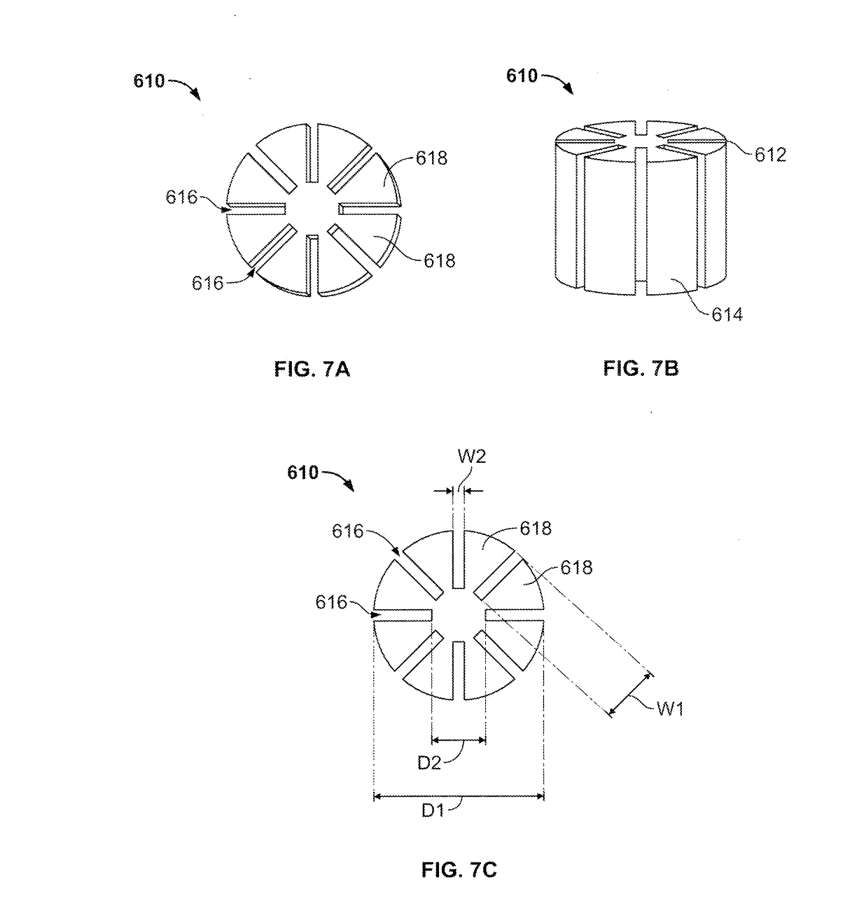

Referring to FIGS. 7A through 7C, an allograft spacer filler 610 includes a first end 612, a second end 614 opposite the first end 612, and a plurality of elongated slits 616 extending from the first end 612 to the second end 614, which form a plurality of flexible arms 618. The spacer filler 610 is made from fully demineralized cancellous tissue, and is adapted to be hydrated with demineralized bone matrix (DBM), bone marrow aspirate (BMA), or any other type of hydrating agent. The spacer filler 610 fits inside a medullary canal of an allograft spine spacer (not shown in the Figures). In this regard, the spacer filler 610 is a “one size fits all” plug, as the slits 616 enable the arms 618 to compress into a small space (i.e., medullary cavities of relatively smaller diameters) or stay expanded in a larger opening (i.e., medullary cavities of relatively larger diameters) without falling out. The slits 616 also help decrease the demineralization time by increasing the surface area of the spacer filler 610. In an embodiment, the spacer filler 610 includes an outer diameter D1 of approximately 15 mm±1 mm, an inner diameter D2 of approximately 5 mm±1 mm, and a height in a range of approximately 13 mm to approximately 17 mm. In an embodiment, each of the slits 616 has a slit depth W1 of approximately 5 mm±1 mm and a slit width W2 in a range of approximately 1 mm to approximately 2 mm. In other embodiments, the spacer filler 610 can consist of different shapes and sizes. In an embodiment, the spacer filler 610 includes six to eight of the slits 616, but it can include more or less than six to eight of the slits 616.

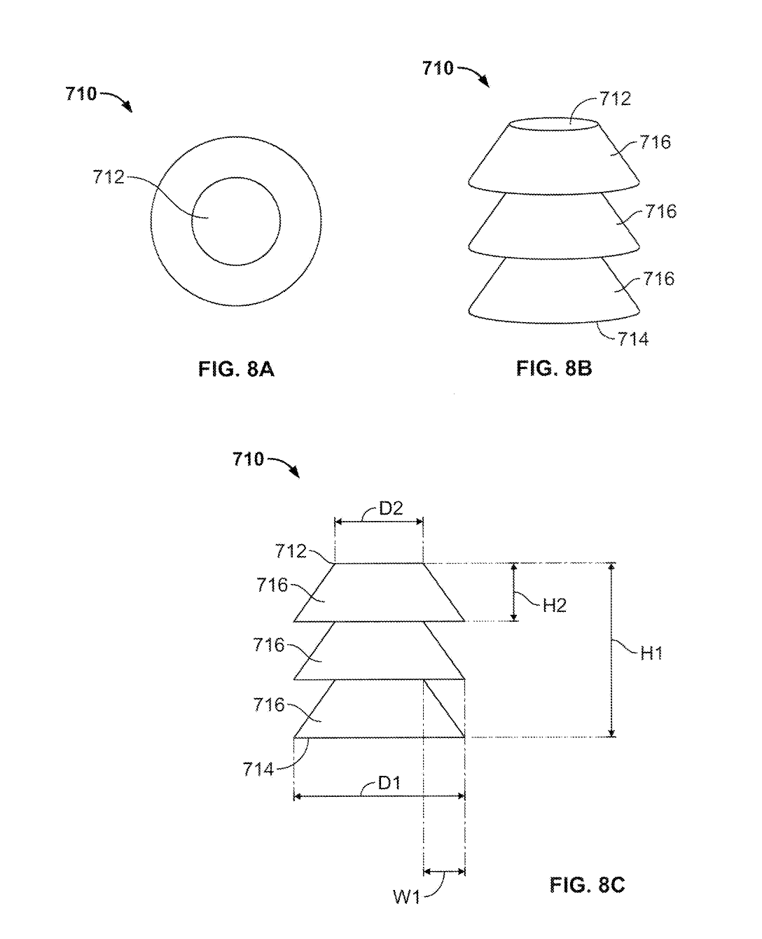

Referring to FIGS. 8A through 8C, an allograft spacer filler 710 includes a leading end 712, a trailing end 714 opposite the leading end 712, and a plurality of flexible wedged sections 716 extending circumferentially between the leading end 712 and the trailing end 714. The spacer filler 710 is made from fully demineralized cancellous tissue, and is adapted to be hydrated with demineralized bone matrix (DBM), bone marrow aspirate (BMA), or any other type of hydrating agent. The spacer filler 710 fits inside a medullary canal of an allograft spine spacer (not shown in the Figures) by inserting the leading end 712 therein. As a result, the spacer filler 710 is wedged into the medullary canal. The wedged sections 716 can fill relatively large sized canals without the spacer filler 710 falling out, while also being able to fold inwardly to fit into relatively smaller sized canals (not shown in the Figures). In an embodiment, the spacer filler 710 has an outer diameter D1 of approximately 15 mm±1 mm, an inner diameter D2 of approximately 7 mm±1 mm, and a height H1 in a range of approximately 13 mm to approximately 17 mm. In an embodiment, each of the wedged sections 716 has a height H2 in a range of approximately 3 mm to approximately 6 mm, and a width W1 of approximately 4 mm. In other embodiments, the spacer filler 710 can consist of different shapes and sizes. In an embodiment, the spacer filler 710 includes three of the wedged sections 716, but it can include more or less than three of the wedged sections 716.

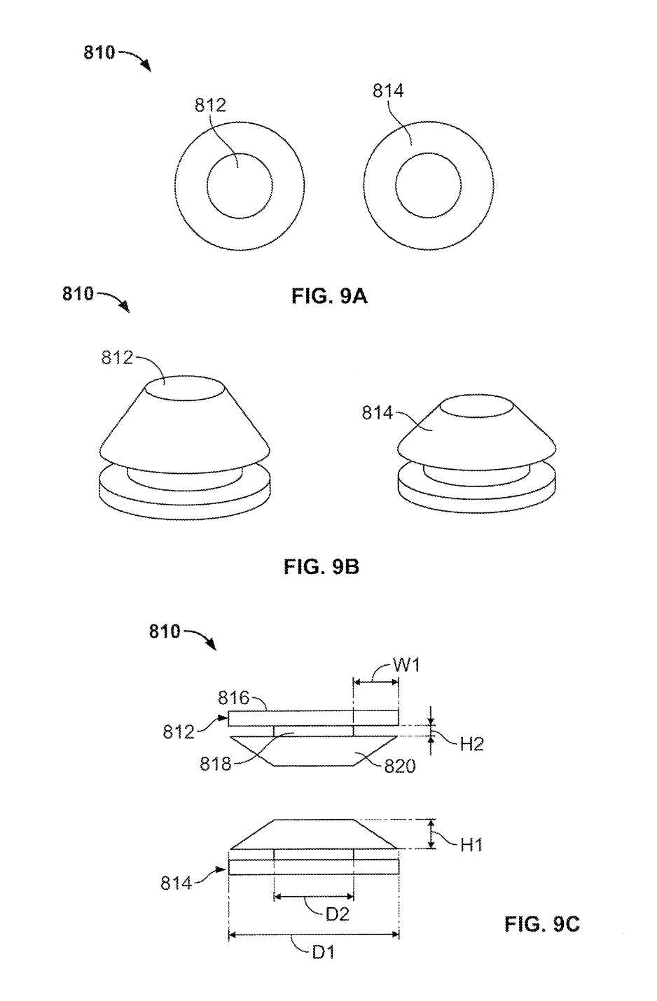

Referring to FIGS. 9A through 9C, an allograft spacer filler 810 includes a pair of plugs 812, 814, which, preferably, are identical in size and shape to one another. Alternatively, the plugs 812, 814 can be different in size and shape. Each of the plugs 812, 814 includes a base 816 with a neck 818 extending therefrom, and a head 820 attached to the neck 818. The spacer filler 810 is made from fully demineralized cancellous tissue, and is adapted to be hydrated with demineralized bone matrix (DBM), bone marrow aspirate (BMA), or any other type of hydrating agent. The spacer filler 810 fits inside a medullary canal of an allograft spine spacer (not shown in the Figures), such that the plug 812 is wedged within the medullary canal at one side of the spacer, while the plug 814 is wedged within the medullary canal at an opposite side of the spacer (not shown in the Figures). More particularly, the plug 812 is inserted within the medullary canal at one side of the spacer, and material of a surgeon's choice can be added into the canal before plugging up the canal at the opposite side of the spacer with the plug 814. In an embodiment, each of the plugs 812, 814 includes an outer diameter D1 of approximately 15 mm±1 mm, an inner diameter D2 of approximately 7 mm±1 mm, and a height in a range of approximately 5 mm to approximately 7 mm. The head 820 has a height H1 in a range of approximately 3 mm to approximately 5 mm, and the neck 818 has a height H2 of approximately 1 mm. In an embodiment, the distance W1 from the neck 818 to an outer edge of the base 816 is approximately 4 mm. In other embodiments, the plugs 812, 814 of the spacer filler 810 can consist of different shapes and sizes.

Each of the spacer fillers 10 through 810 described above and their associated features can be manufactured by any appropriate means known in the art, e.g., cutting, sawing, milling, lathing, boring, etc. While the spacer fillers 10 through 810 can be used with spinal spacers, they can be adapted for use in other applications where allograft implants are used.

It will be understood that the embodiments of the present invention described herein are merely exemplary and that a person skilled in the art may make many variations and modifications without departing from the full spirit and the scope of the embodiments described herein. Accordingly, all such variations and modifications are intended to be included within the scope of the present invention.

We claim:

1. An allograft spacer filler, comprising a body made from demineralized cancellous bone and including a first end, a second end opposite said first end, a longitudinal axis extending from said first end to said second end, a core region located proximate to said longitudinal axis and extending from said first end to said second end, and a peripheral region located distal from said longitudinal axis and extending from said first end to said second end, said peripheral region including a plurality of spaced-apart projections extending laterally outwardly from said core region, each of said projections being compressible in a radial direction toward said core region to facilitate the insertion of said body into a cavity of relatively smaller diameter, said projections having shape memory such that they are radially expandable after their compression in a radial direction.
